(19) United States
(12) Patent Application Publication
(10) Pub. No.: US 2010/0036452 A1
(43) Pub. Date: Feb. 11, 2010

(54) Modulation of neural traveling waves

(76) Inventors: Bruce Gluckman, State Gllege, PA (US); Kristen Richardson, Fairfax, VA (US); Steven Schiff, State Gllege, PA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/666,654

(22) PCT Filed: Nov. 2, 2005
(86) PCT No.: PCT/US2005/039891

§ 371 Date: May 14, 2009

Related U.S. Application Data
(60) Provisional application No. 60/623,859, filed on Nov. 2, 2004.

(51) Int. Cl. A61N 1/36

(52) U.S. Cl. 607/45

(57) ABSTRACT

The present invention provides devices and methods for modulating the properties and propagation of traveling waves of electrical activity in neural systems. Such modulation is useful for a variety of purposes, including the control and containment of epileptic seizure activity, for treating mental disorders, movement disorders, sleep disorders, pain, and other disturbances and illnesses associated with neural systems. In addition, the devices can be used to modulate sensory and other stimuli experienced by a neural system, as well as any other normal neural activity. Neural prosthetic devices incorporating the methods of the present application have a wide range of applications and use in medicine, psychiatry, behavioral psychology, research, and other disciplines that address and treat disturbances in neural systems.

This application claims the benefit of U.S. Provisional Application No. 60/623,859, filed Nov. 2, 2004, which is hereby incorporated by reference in its entirety.

### BRIEF DESCRIPTION OF THE DRAWINGS

FIG. 1. Relationships between model parameters: For a given threshold (θ) and relative time constant (ε), there are either 2 or 0 solutions of speed (c) and width (a) of the supported pulse. The solutions in the gray region are probably unstable and unlikely to be observable in the experimental system. An example of a stable traveling pulse is shown below (a=0.10, θ=0.25).

FIGS. 2 (A, B, and C). Schematic of global field (left) and local field (right) experiments. A bipolar stimulating electrode (SE) placed approximately in layer V/VI is used to initiate traveling wave activity that propagates parallel to the pial surface. Recording electrodes are placed in layer II/III near the initiation site (R1) and far from the initiation site (R2, R3) to track the propagating activity. A: Large Ag/AgCl electrodes (FE1, FE2) embedded in the interface chamber floor are used to apply the global electric field. B: A small Ag/AgCl cylindrical pellet (FE1) placed close to the pial surface is used to apply a local electric field and a reference electrode is placed in the artificial cerebrospinal fluid layer far from the slice. C: Direction of a positive field from FE1 in relation to pyramidal cells in layer V. The extended structure of the dendritic arborization of the cells in this layer allows for field polarization.

FIGS. 3 (A and B). Global Field results. A: Extracellular recordings made during the positive (top traces), zero (middle traces) and negative (lower traces) electric field application. The activity wave arrives at R2 earlier during positive field (top) and later during the negative field (lower) relative to the zero field (middle) application. B: Wave speed (bottom) normalized by baseline velocity and Failure rate (top) as a function of field amplitude for four separate experiments. As the field amplitude becomes more positive, the speed increases, and as the field amplitude becomes more negative, the speed decreases. As the field amplitude becomes increasingly negative the rate of failure of the activity to propagate increases to complete failure.

FIGS. 4 (A and B). Local field results. A: Extracellular recordings made during the positive (top traces), zero (middle traces) and negative (bottom) local field application. The time delay for activity to travel to R2 (the left edge of the parallelogram) from R1 (black vertical line) varies with applied field amplitude. Alternatively, travel time is independent of field amplitude between R2 (left edge of parallelogram) and R3 (right edge of parallelogram) where the electric field is smaller and not aligned with the neurons. B: Wave speed (bottom) normalized by baseline velocity as a function of field amplitude for two experiments. In the two top traces, the field is applied (locally aligned with the neurons) between R1 and R2. In the bottom two traces, the field is applied between R2 and R3. In either case, within the applied field, speed increases with increasing field amplitude, and is independent of amplitude outside the electric field.

### DESCRIPTION OF THE INVENTION

The present invention provides devices and methods for modulating the properties and propagation of traveling waves of electrical activity in neural systems. Such modulation is useful for a variety of purposes, including the control and containment of epileptic seizure activity, for treating mental disorders, movement disorders, sleep disorders, pain, and other disturbances and illnesses associated with neural systems. In addition, the devices can be used to modulate sensory and other stimuli experienced by a neural system, as well as any other normal neural activity. Neural prosthetic devices incorporating the methods of the present application have a wide range of applications and use in medicine, psychiatry, behavioral psychology, research, and other disciplines that address and treat disturbances in neural systems.

As described in more detail below, the present invention arises from the discovery that traveling electrical waves in complex neural systems can be controlled by the directed application of electric fields and currents. In an embodiment of the present invention, methods and devices are provided for modulating the propagation speed and/or properties of a traveling wave in a mammalian neural system, comprising applying an electric field to the neural system that is experiencing a traveling wave of electrical activity and that is effective for modulating the propagation speed and/or properties of this traveling wave in a mammalian brain. Wave trajectories and velocities in the neural system can be dynamically controlled by applying electrical fields of defined orientation, amplitude, and gradient.

Traveling waves in excitable media are commonly observed in physical, chemical, and biological systems. The present invention provides methods and devices for modulating their properties. Modulation can be achieved in several different ways. For example, it can be used to contain or block a traveling wave. Certain waveforms initiate in one region of the system (e.g., a “focus”), and then propagate outwardly through it, changing the properties of the regions which surround it. When the waveform is associated with epilepsy, or other anomalous waveforms (e.g., associated with Parkinson's disease tremors), wavefront propagation can have progressively adverse effects on the neural system and the corresponding behavior of the elements it controls. By administering an electric field to a region outside the focus of electrical activity, the present invention provides the means to alter or change the characteristics of the wave, thereby preventing its spread to other areas, and alleviating or treating symptoms otherwise associated with it.

Traveling wave modulation, however, is not restricted to controlling the spread of electrical activity that originates in one region of a neural system. Applied fields can also be applied in regions that are experiencing a waveform activity. In these cases, the field can be utilized to modulate the wave activity, e.g., by changing its speed, direction, intensity, and other characteristics. This is useful for interacting with any neural system activity, including sleep, mental states, motor activity, sensory activity, and generally any activity (normal or aberrart) controlled by the neural system.

A neural system in accordance with the present invention can be any ensemble of neurons, and can include non-neuronal cell types, such as glia, Schwann cells, microglia, oligodendrocytes, and astrocytes. Neuronal cells can be in the form of mature, stem, progenitor, and/or tissue culture cells, as well as including cell-electrode interface devices and the like. Cells can be coupled electrically, chemically, or combinations thereof.

The neural system can be an entire brain, ganglia, nerve, etc., or it can be a region or portion of it. Any animal source of neuronal material is suitable, including neural systems of invertebrates, such as mollusks, arthropods, insects, etc., vertebrates, such as mammals, humans, non-human primates, great apes, monkeys, chimpanzees, dogs, cats, rats, mice, etc.

The charge and amount of electric field that is applied to the neural system depends upon the purpose of the field intervention. As shown in the examples below, both positive and negative fields can be applied to a neural system with different effects on the traveling wave. For example, the velocity (customarily expressed as millimeters, centimeters, or meters per second) of traveling waves can be increased by making the field amplitude more positive with respect to the surface of the brain. This can be useful to enhance a neuronal activity, increase the system's response to a stimulus, or facilitate other excitatory events.

Increasingly negative field amplitudes with respect to the surface of the brain can be used to reduce and block wave propagation, e.g., to contain an epileptic seizure; block the symptoms of a movement disorder; and/or to reduce any undesirable neuronal activity (e.g., to block or reduce sensitivity to pain, and visual, auditory, and somatosensory stimuli). The characteristics of the electric field necessary to achieve a desired effect (e.g., to improve a mood disorder) can be determined empirically, where different field polarities with differing characteristics can be applied until a therapeutic effect is achieved.

The applied electric field can be monophasic (i.e., negative or positive) or it can be a mixture of different fields that vary in polarity, frequency, etc., depending on the particular disorder which is to be treated or the desired neural effect. It may be therapeutically useful to apply a mixture of polarities, e.g., to prevent break-through seizures; to treat regions that have already been sensitized or desensitized by previously applied fields; and to define a pattern of activity in a region of the brain through which a traveling wave is about to propagate.

Any suitable applied field can be administered to the neural system. An applied field can be continuous (e.g., where the field is turned on for a time period, and then turned off); intermittent; and/or comprised of a pulse or train of pulses. The fields can be regular (e.g., sinusoidal) an/or of an arbitrary form. The field amplitude, frequency, intervals between application of fields, and field duration can also be varied depending on the purpose to be achieved.

In preferred embodiments of the present invention, the field amplitude is applied in an amount that is sub-threshold to the neurons in the system. By the term “sub-threshold,” it is meant that the amount of applied field does not reliably, with 100% probability, initiate new action potentials within the neural system. In contrast, the application of a supra-threshold stimulus reliably, with a high degree of probability, results in neuronal firing. A sub-threshold field is, for example, less than 100 mV/mm, preferably 50 mV/mm or less, more preferably, 25 mV/mm or less, such as 20 mV/mm, 15 mV/mm, or 10 mV/mm. The sub-threshold field refers to the potential generated at the level of the target neurons. The amount of potential actually produced by the field electrodes is less important that the field perceived by the target neurons. It is the generated field sensed by the neurons that determines whether a stimulus is sub- or supra-threshold.

A sub-threshold field does not significantly polarize the individual neurons in the system. Although certain regions of the neuronal membranes of individual neurons may be differentially affected by the field to produce localized elevated concentrations of negative or positive ions (depending on their orientation within the field), the total charge on the neuron is not substantially changed. While not wishing to be bound by any mechanism, such a sub-threshold field has the effect of changing the excitatory threshold of the neuron such that it requires a larger or smaller depolarizing stimulus to initiate an action potential. In contrast, Fischell et al. in U.S. Pat. No. 5,938,699 describe applying functionally depolarizing stimuli which lead to the flow and net accumulation of positive ions within the cell, raising the net membrane potential of the cell close to the threshold value. To the extent that the applied field stimuli of the present invention do not cause net depolarization of the cell as described by Fischell, the stimuli can be characterized as functionally “hyperpolarizing” to indicate that the neurons are pushed further away from their threshold level.

The electric field in preferred embodiments of the present invention can produced using low frequencies, e.g., from about 100 Hz or less, 50 Hz or less, 25 Hz or less, 10 Hz or less.

To contain and/or block a traveling wave, electric fields which are sub-threshold (also functionally “hyperpolarizing”) to the neurons in the neural system are preferably applied. Such a sub-threshold electric field at the surface of the neocortex is generally an electric field which is negative at the surface of the cortex with respect to deeper layers, e.g., which is effective to raise the threshold to fire of individual neurons in the neural system, reducing the probability that such a neuron would fire in response to a stimulus (e.g., the electrical or chemical signal from neighboring neurons), thereby blocking or reducing wave propagation. In other regions of the brain, such as the hippocampus of the temporal lobe, a field positive at the surface with respect to deeper layers may hyperpolarize neurons because the neuronal geometry of the excitatory cells in the hippocampus can here be inverted with respect to the neuronal geometry of neurons from the neocortex. When sub-threshold stimuli are applied for the purpose of blocking or containing a traveling wave, the stimuli are preferably applied outside the region of activity and ahead of the traveling wavefront.

Because of the geometry of the brain and the individual neurons which comprise it, the polarity of the sub-threshold field will vary depending on the position of the electrodes and the target region to which the field is directed.

The present invention also provides methods and devices for controlling the propagation patterns of traveling waves in neural systems. In this embodiment of the present invention, an applied field is utilized to control a wave that is propagating through the neural system. As shown in the examples, electric field amplitude can be used to increase or decrease wave velocity. The direction of a traveling wave can also be altered by changing the position of the applied field with respect to its “center of mass.”

The present invention relates to the modulation of traveling waves, irrespective of the modulatory mechanism. Although the disclosure may refer to “sub-threshold” and “hyperpolarizing electric fields,” the present invention is not bound to any mechanism through which wave modulation and efficacy is achieved. Neural systems include other significant elements, for example, extracellular spaces, as well as glial cells that react to electrical and chemical signals, which produce and secrete their own chemicals that are physiologically active in the neural system, and which are also involved in the dynamical neuronal activity of the system. In addition, such fields affect the efficacy of synaptic transmission, often from field orientations which differ from those which are optimally suited for neuronal firing threshold modulation. Thus, the field stimulus may be defined in terms of its effect on neurons, but this does not limit its effect to neurons only, nor does it imply that neurons are solely responsible for the palliative results.

The field can be produced using any suitable type and/or arrangement of electrodes (“field electrodes”) that are effective to produce a field with the desired polarity. As indicated above, in order to modify the progression and spread of the traveling wave, the field is applied outside of the region experiencing its electrical activity. Very generally, a field can be produced using a simple DC circuit comprising a power source (e.g., a battery) to produce current or voltage; a controller (e.g., a signal generator, or an adaptive controller which permits the current or voltage to be changed in response to system feedback) to drive current or voltage of desired characteristics through the system; and at least two strategically placed electrodes (a current-source and a current-sink).

The controller can further comprise electronics for monitoring and limiting the stimulus strength and hence the electrochemical potential developed at the electrodes within safe limits. Power sources are conventional, and can be fixed or adjustable, where the field amplitude, frequency, etc., can be manually or adaptively controlled. In addition, the field-producing device can also comprise a detector which measures the current and/or voltage which is produced by the power source. Examples of devices that can be used to generate fields in accordance with the present invention include, e.g., Gluckman and Schiff (U.S. Pat. No. 6,873,872).

The electrodes are typically positioned in an arrangement that the current flow between them passes through the target region. A simple arrangement can comprise a deep or depth electrode (current sink or source) in the target region, and a second electrode (current sink or source) outside of the region, e.g., on the brain surface; in a fluid space within the system; outside the system (e.g., outside the brain on the chest); etc. Another arrangement is to have an electrode(s) on the surface as a current sink or source which is positioned over the target neural region so that the current spreads directly into it.

More than one electrode can be employed, e.g., where an addressable array of electrodes (including micro-electrodes) is used to deliver the electric field. An example of such an electrode array includes a sheet comprising multiple electrodes that can be placed over the neocortex in the subdural, subarachnoid, or epidural spaces, or within the sulci of the brain. By making the array addressable, the position of the electric field can be adjusted depending upon the position within the neural system region where the field is to be delivered. This can be especially advantageous since it adds flexibility to the device, especially when a traveling wavefront is being tracked and its position varies with time.

Addressable electrode arrays are especially useful for modulating traveling waves in accordance with the more general concepts of the present invention. This includes generating complex waveforms and differentially modulating traveling waves in different parts of the brain. For instance, different electrode sets can be utilized to either simultaneously or consecutively to modulate traveling waves in different parts of the brain.

The field (producing) electrodes, methods and devices of the present invention can also incorporate additional sets of electrodes for observational purposes and to ascertain the dynamic properties of the neural system. These may be bundled with the field electrodes or be used as separate units.

“Field-monitoring electrodes” can be utilized to monitor the actual electrical field that is experienced by the neural system. Although a current of a set value may be driven through the field electrodes, the actual observed current in the system will differ, depending, e.g., on its conductive and resistive properties, and the geographical and spatial placement of the electrodes. The field-monitoring electrodes can be placed in the system within the region where the stimulus is delivered to make such observations. Signals from the field-monitoring electrodes may be processed using conventional algorithms to reduce artifacts and derive an authentic value for the applied field in the neural system.

Field-monitoring electrodes can also be used to monitor the junction potential of the field electrodes to avoid exceeding the safety limits of stimulation. For example, high junction potentials can produce tissue damage. For junction potential monitoring, an additional electrode can be utilized which is comprised of a material, such as platinum, whose own junction potential does not significantly vary as ion concentrations change over time.

The system activity can be measured in any suitable way, including using, e.g., optical, metabolic, chemical, electrical, behavioral, etc., measurements for assessing the status of the system and its dynamic properties. Behaviors, or other products of a neural system (e.g., hormones, growth factors, neurotransmitters, ions, etc.), can be detected and used as a measure of system activity) (i.e., neuronal activity). For instance, if a purpose is to block or reduce tremors, then the system activity can be monitored by assessing their frequency and magnitude.

“Measurement electrodes” are particularly useful for measuring the electrical activity of the system. Neurons typically display variations in their membrane potential, such as action potentials, depolarizations, and hyperpolarizations. These changes in the membrane potential can be utilized as a measure of electrical activity, e.g., by monitoring intracellularly in a single neuron, or extracellularly, the electrical activity of a single neuron or the activity of an ensemble of neurons as local field potentials, electrocorticogram, or electroencephalogram. The electrical activity which is measured or assessed can be a subset of the total activity observed in the system, e.g., a particular frequency band of the full neural signal. For example, the measuring electrode may be capable of detecting various types of activity, including spontaneous neuronal firing, slow burst activity, and background noise, as well as fast frequency epileptic seizures. However, filters can be utilized to extract the frequencies and events of interest.

Typically, field-monitoring and measurement electrodes can be operably connected to measurement electronics (e.g., an amplifier) for preamplifying and amplifying the electrical signal, as well as discriminating the electrical signal from the applied field (stimulation artifact removal).

The device can also include a feedback controller, e.g., as described in U.S. Pat. No. 6,873,872, for modulating the applied field in response to changes in electrical activity in the system. The general plan for a feedback controller includes field-monitoring electrodes operably connected to pre-amplification electronics. These convert a physical quantity, such as the electrical activity as expressed by variations in extracellular field potential, to an electrical signal. The next step is some level of signal conditioning, including filtering, amplification, and stimulus artifact removal. The conditioned measurement signal is then fed to a controller, i.e. one that takes in the measurement and outputs a prescription for the stimulation. This could be done with analog or digital circuitry. More often this is done with digital circuitry, which could include combinations of analog to digital converters (ADC), digital signal processors (DSP), microcontrollers, and digital to analog converters (DAC). Both DSPs and microcontrollers can be used to process digital signals and compute output values. The output values (prescribed control signal) is then converted back to an analog voltage which is used to control the output of the signal generator, which then drives current between or amongst the field electrodes.

Any effective electrodes can be used for the recording, sensing, and field electrodes, including, e.g., metal, steel, activated iridium, platinum, platinum-iridium, iridium oxide, titanium nitride, silver chloride, gold chloride, etc., where the electrode can be insulated by glass or lacquer, as well as silicon microelectronics, including tetrode or other multielectrode arrays or bundles, multichannel and ribbon devices. Typically, the electrodes can have relatively large tips with low resistance to detect activity from a number of neuronal elements within the neural system. Smaller tipped electrodes can be used for monitoring activity from single neurons or smaller populations. Activity can be measured from one or more electrodes, preferably two or more. In some cases, it may be desired to record from several regions of the neural system in order to characterize its activity. Recordings of intracellular, extracellular, or a combination thereof, can be analyzed separately, or together. The electrodes can be AC- or DC-coupled.

For certain purposes, iridium oxide type electrodes may be preferred since they are relatively nortoxic to cells, as well as being effective carriers of high current and charge densities. An activated iridium or iridium alloy wire can be used, or a metal substrate, such as noble metal (e.g., Au, Pt, or PtIr), ferrous steel alloy, stainless steel, tungsten, titanium, silicon microprobe, etc., or other suitable substrate, can be coated with a film of iridium oxide to produce an effective electrode. Any suitable method to prepare the coating can be used, including, but not limited to, an activation process (e.g., Loeb et al., J. Neuro. Sci. Methods, 63:175-183, 1995; Anderson et al., IEEE Trans. Biomed. Eng., 36:693-704, 1989) to form activated iridium oxide films (AIROFs), thermal decomposition (Robblee et al., Mat. Res. Soc. Symp. Proc., 55:303-310, 1986) to form thermal iridium oxide films (TIROFs), reactive sputtering (15) to form sputtered iridium oxide films (SIROFs), electrodepositing (Kreider et al., Sensors and Actuators, B28:167-172, 1995) to form electrodeposited iridium oxide films (EIROFs), (Meyer et al., IEEE Transactions On Neural Systems And Rehabilitation Engineering, 9: 2-11, 2001), etc.

As mentioned, a device of the present invention can be utilized for suppressing the propagation of epileptiform activity in a mammalian brain, comprising: applying an amount of a sub-threshold (as well, as functionally hyperpolarizing electric field) to a mammalian brain experiencing ongoing epileptiform activity, wherein the electric field is applied to a region of the brain which is not experiencing epileptiform activity and the amount is effective for suppressing the propagation of epileptiform activity in said mammalian brain. Various foci of epileptiform activity are well-characterized, including, e.g., hippocampus, or any neocortical region of the brain.

In one embodiment of the present invention where the purpose is to control or contain epileptic traveling waves, one or more longflat electrode strips can be inserted through an occipital entrance hole such that it contacts the long axis of the hippocampus surface in the temporal horn of the lateral ventricle. A round electrode (e.g., a single depth electrode with one or more suitable high current contacts) can also be utilized, e.g., by placing it within the long axis of the hippocampus in order to produce a radial electric field. As discussed above, a deep electrode can also be comprised of a plurality of addressable electrodes at different distances proximal to the occipital point of insertion. This configuration enables the electric field to be applied differentially, depending on the position of the wavefront of the traveling wave. After initiation of the epileptiform activity, the device can be programmed or manually changed to apply an electrical field across any point on the electrode array to anticipate the progressing wave and/or to create a fail-safe type mechanism, where the field is produced progressively (i.e., temporally and spatially) distant to the focus in order to attempt to block wave progression.

Useful electrode strips include non-polarizing biocompatible electrodes embedded in silastic sheets with sealed electrode-lead connections, similar to those used for cochlear implants, e.g., a Clarion Cochlear Implant, comprising iridium oxide electrodes sealed within a curved silastic silicone elastomer sheath.

An electrical field can be produced in any target region of the brain, including but not limited to,

cortical zones: including, primary visual, auditory, somatosensory, or motor cortex; unimodal association zones, such as visual, auditory, somatosensory, or motor; heteromodal association zones, such as temporal cortex, temporoparietal cortex, posterior parietal, prefrontal and frontal cortex; paralimbic zone, such as orbitofrortal cortex, insula, temporal pole, parahippocampus cortices, and cingulated cortex/complex; limbus zone, such as septal nucleic, piriform cortex, amygdala. hippocampus, hypothalamus, epithalamus and Habenula, reticular formation, formix, limbic striatum, substantia innominoto, and globus pallidus;

basal ganglia: including striatum, such as neostriatum, caudate, and putamen; limbic striatum, such as nucleus accumbens and olfactory tubercle

diencephalon: including thalamus, epithalarnus, subthalamus, hypothalamus, and internal capsule;

white matter tracks: including corpus callosum, internal capsule, external capsule;

spinal cord: including dorsal columns, medial and lateral lemnisci, pyramidal tracts, motor pools;

brain stem: including midbrain, such as substantia nigra and red nucleus; pons, such as locus ceruleus; and medulla oblongata, such as inferior olivary complex.

In addition to epilepsy, the devices and methods can be used to contain and/or modulate traveling wave activity associated with any neurological or psychiatric disorder, especially disorders associated with anomalous oscillatory or pulsating activity. For example, thalamo-cortical dysrhythmia has been associated with various neuropsychiatric syndromes. See, e.g., Llinas et al., Trends Neurosci. 2005 June; 28(6):325-33. Disorders that can be treated in accordance with the present invention include, e.g., schizophrenia, depression (unipolar or bipolar), Parkinson's disease, anxiety, obsessive-compulsive disorder (OCD), attention deficit hyperactivity disorder, etc., where the electric field is applied to the particular brain region outside the source of the anomalous wave, e.g., cortex, hippocampus, thalamus, etc. For example, Parkinson's disease is characterized by decreased activity in cells that produce dopamine. Patients with the disease experience tremors, rigidity, and difficulty in movement. Patients with Parkinson's disease can be treated by applying an electric field outside the originating source of electrical activity in an amount effective to ameliorate one or more symptoms of the disease.

Disorders and the originating region of the wave activity include, but are not limited to: Parkinson's diseases (e.g., thalamus; subthalamic nucleus; internal or external segment of globus pallidus; substantia nigra; within the thalamocortical pathways); sleep disorders (e.g., thalamic neurons; cortical regions; brainstem neurons including reticular activating system, raphe nuclei, locus ceruleus); migraine (e.g., cortical regions); dystonia; pain; depression; schizophrenia; mood disorders (e.g., anterior cincugate, internal capsule, septo-hippocampal system (Hajos et al., Neuropsychopharmacology. 2003 May; 28(5):857-64)). An electrical field in accordance with the present invention can be placed in a region of the brain which is effective to modulate the properties of anomalous waves of activity.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure of U.S. Provisional Application Ser. No. 60/623,859, filed Nov. 2, 2004 is hereby incorporated by reference herein.

From the foregoing description, one skilled in the art can easily ascertain the essential characteristics of this invention and, without departing from the spirit and scope thereof, can make various changes and modifications of the invention to adapt it to various usages and conditions.

### EXAMPLES

Wilson and Cowan developed a set of integro-differential equations to form a continuum model of cortex which demonstrated traveling waves. This model was recently modified to reflect traveling pulse propagation in disinhibited neocortex. The model predicts that threshold determines several dynamical properties of the traveling wave. Specifically, at low threshold waves travel quickly, and at high threshold waves travel slowly. The model is described as

$$\frac{\partial u ( x , t )}{\partial t} - u ( x , t ) = {\int }_{- ∞}^{∞} w ( x - {x}^{′} ) P ( u ( {x}^{′} , t ) - θ ) d {x}^{′} - v ( x , t ) \\ \frac{1}{ɛ} \frac{\partial v ( x , t )}{\partial t} + β v ( x , t ) = u ( x , t ) . \qquad ( 1 )$$

The variable u represents the activity, which is the fractional firing rate of the local neurons. Activity at point x at time t,u(x,t), is a function of the activity of the whole population with spatial connectivity w (here chosen as exp(−|z|)/2). The threshold of the excitable system is reflected in the activity function P which depends on the activity of points relative to this threshold, (u(x′,t)−θ). We choose P to be the Heaviside function. Synaptic input therefore comes only from cells that have activity greater than threshold. A recovery variable, v(x,t), has been added to account for activity accommodation and adaptation due to such processes as sodium channel inactivation, potassium channel activation (voltage and calcium sensitive), and other intrinsic mechanisms which contribute to refractoriness in the wake of activity. The parameters reflects the relative time constant for changes in v versus u and is typically small.

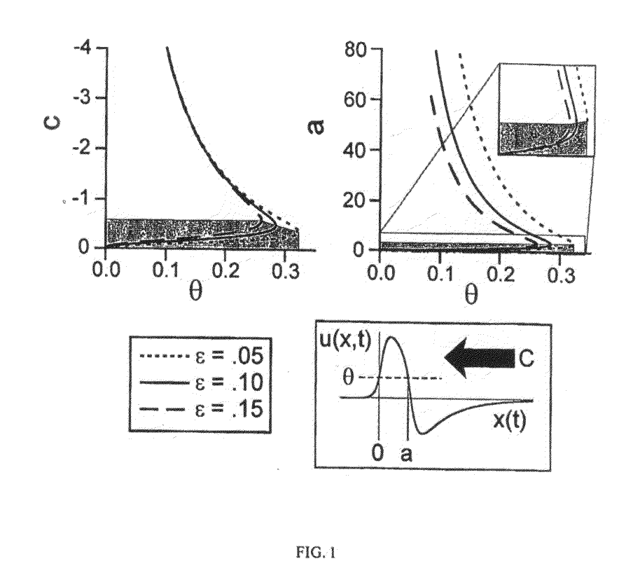

We assume a traveling wave solution, u(x,t)=u(x−ct)=U(z), such that equation (1) collapses to −c²U″+cU′−εU=c[w(z)−w(z−a)]. Boundary such that U goes to zero at positive and negative infinity, and is above threshold θ between 0 and a (see FIG. 1 inset): that is U(0)=U(a)=θ,U(±∞)=0.

The solution is partitioned into three regions: (1) z<0, (2) 0≦z≦a, and (3) z>a and can be written as U(z)=Aᵢe^{z}+Bᵢe^{-z}+Cᵢe^{λz}, where i denotes the solution region (1, 2, or 3). By matching the three sets of coefficients at the boundaries, U(0) and U(a), we numerically determine the relationships between the four parameters of the system: relative time constant (ε), speed (c), pulse width (a) and threshold (θ) shown in FIG. 1. Pulse speed and width are shown as a function of threshold for several values of ε (solid and dashed lines).

There exist two solutions for speed and width for a given pair of threshold and relative time constant values. The smaller width and speed solution (FIG. 1, gray region) is thought to be unstable. Such instability for a smaller wave in bistable solutions was noted by Amari for a field model of stationary pulses of activity in a network of neurons (S. Amari, Biol. Cybernetics 27, 77 (1977)) and found by Rinzel and Keller (Biophys. J. 13, 1313 (1973)) for analogous problems in a Fitzhugh-Nagumo model of traveling pulses in axons At sufficiently large values of threshold, both solutions disappear, and all pulses decay. Note that the solutions disappear at finite values of width and propagation speed. At very small θ, we cannot calculate the stable branch of the solutions due to instabilities in the numerics. The unstable solutions in the gray region (FIG. 1) are unlikely to be observable in the experimental system.

Thus, these equations predict that speed and width depend on threshold such that for the stable solution at low threshold (small θ) the pulses are wide and fast (large a and c, respectively), and at higher threshold (large θ) the pulses are narrow and slow (small a and c). At sufficiently high threshold propagation will fail at non-zero speed and width.

We therefore hypothesized that we could speed up, slow down and block propagating neural activity in cortex with the application of electric fields. Furthermore, we predicted that we would affect wave propagation either globally, over the whole slice, or locally, in a localized region of the slice, by changing the geometry of the applied field.

Transverse neocortical slices (400 μm thick) were prepared from adult male Sprague-Dawley rats (age p 26-p 68) and bathed in artificial cerebrospinal fluid (aCSF: 130 NaCl, 1.25 NaH₂PO4, 3.5 KCl, 23.9 NaHCO₃, 1.1 MgSO₄, 10 Dextrose, 1.23 CaCl₂, in mM) containing low doses (4.7-8.3 μM) of picrotoxin, a blocker of γ-aminobutyric acid A (GABA_{A}) inhibitory synapses. Previous work on propagation of activity in neocortical slices bathed in low doses of picrotoxin revealed that layer V neurons are necessary for supporting initiation and transmission of activity across the slice (ⁱ). The pyramidal neurons in layer V are large and asymmetric and presumably easily polarizable with applied electric fields. Therefore this experimental system is amenable to electric field modulation for altering propagation dynamics.

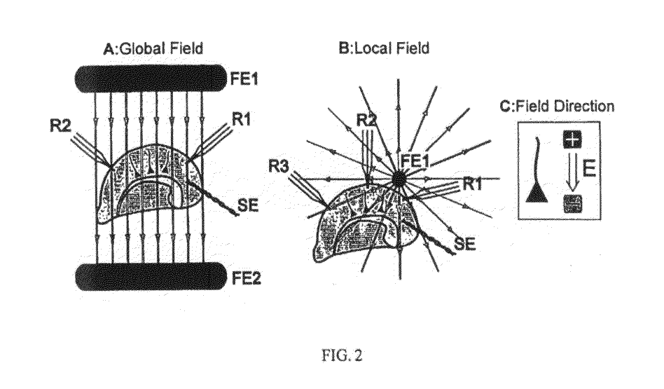

A bipolar stimulation electrode (FIG. 2, SE) was placed in layer V/VI at one end of the slice to initiate epileptiform bursts that propagated parallel to the pial surface (A. E. Telfeian, B. W. Connors, Epilepsia 39,700 (1988); R. D. Chervin, P. A. Pierce, B. W. Connors, J. Neurophysiol. 60, 1695 (1988)). Only slices with stable waveform initiation were used. An electric field was applied across the entire slice (Global Field, FIG. 2A) or across a small localized region of the slice (Local Field, FIG. 2B) using custom-built voltage- or current controlled circuitry and nonpolarizing (Ag/AgCl) electrodes (FIGS. 2A and 2B: FE1, FE2). We define a positive electric field as one oriented from dendrite to soma (FIG. 2C). Such a field decreases the somatic transmembrane voltage difference from rest, which brings the neuron closer to firing threshold.

Wave propagation was initiated with a short (0.15 ms) current pulse (0.1-1.0 mA) applied through an RC circuit to the bipolar stimulation electrode. The amplitude of the pulse was fixed for the duration of the experiment at a level determined to reliably initiate propagating activity. A waveform generator (Hewlett-Packard 33120A) and custom electronics were used to generate and apply a multiphasic periodic electric field (100-125 mHz), consisting of four phases: (1) positive, (2) zero, (3) negative and (4) zero amplitude DC fields. Each phase corresponds to the application of a constant field for duration of 2-2.5 seconds. Transitions between the phases were smoothed with a low-frequency half sinusoid between each phase. To examine neuronal propagation speed as a function of applied field, waves were initiated by stimulating during the various phases of the waveform. Propagation speed was determined with approximately 15 repetitions at each field amplitude for each phase.

Double-barreled glass micropipette electrodes filled with 0.9% NaCl were used to differentially record local extracellular field activity in layers II/III using custom-built pre-amplifiers (gain=10) and an amplifier bank (DAGAN) with bandpass filter settings (1 Hz-1 kHz) and gain of 200. In the global field experiment, extracellular activity was recorded in two places in layer II/III: near the wave initiation site and more distant from the initiation site (2-10 mm interelectrode distance). In the local field experiment, three recording electrodes were placed to record the propagating activity across the surface of the slice with the local field placed between either the first or second pair of electrodes. Propagation speed was determined by the transit time between electrode pairs. In order to account for experimental drift, baseline speed as a function of time was determined with a polynomial regression fit during the zero field phases. Speed as a function of field is presented as a percentage of this baseline speed.

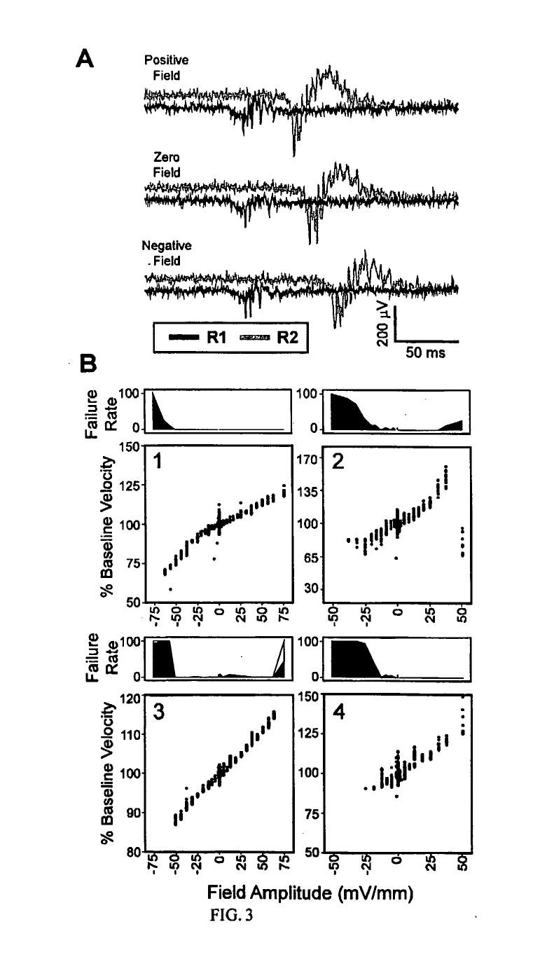

We observed modulation of propagation speed with global electric field in 25 of 25 slices. Examples of raw data are shown in FIG. 3A. For each set of recordings the black traces were recorded at R1 near the initiation site and the light gray traces were recorded 2 mm along the propagation path at R2. The speed between R1 and R2 is field dependent: faster during positive field (top traces) and slower during negative field (bottom traces) relative to the zero field (middle traces). The speed normalized by the baseline speed is shown as a function of field amplitude for four typical experiments in FIG. 3B (larger graphs).

Sufficiently large negative fields caused propagation failure. The rate of failure depended on the amplitude of the suppressive field (FIG. 3B, smaller graphs). Failure rate was defined when waves passed R1 but did not reach R2.

In some experiments, very high positive (excitatory) field application caused wave initiation prior to stimulation. This caused apparent propagation failure of the stimulated waves due to refractoriness. This premature wave initiation at high positive fields could also be followed by aberrantly slow stimulated waves (FIG. 3B, second experiment), and occasional initiation failure (FIG. 3B third experiment).

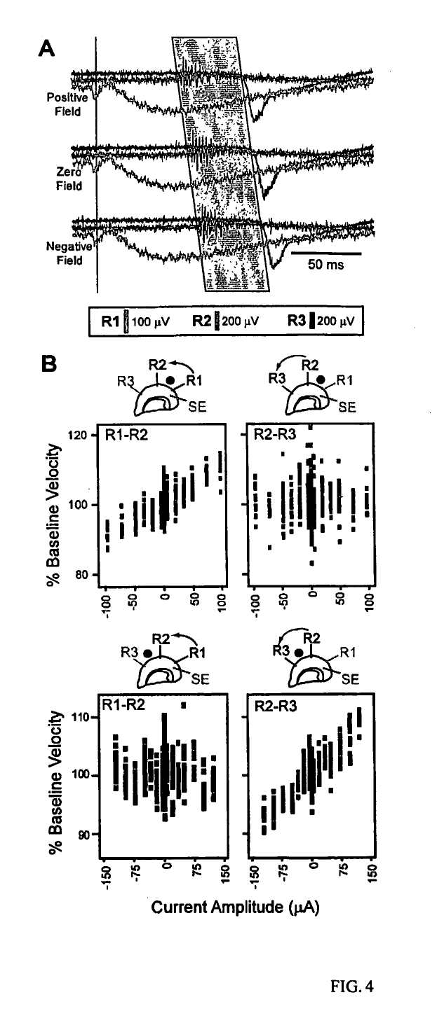

We observed modulation of propagation speed in 18 of 20 slices exposed to local electric fields. In these experiments, modulation was observed only in the region spanning the local field. In the other 2 of 20 slices, no clear speed modulation was observed. Examples of raw data are shown in FIG. 4A. The field, which falls off radially, was large and aligned with the neurons in the region spanned between R1 and R2 (see FIG. 1B), and small and misaligned with the neurons between R2 and R3. The time to travel between R2 and R3 is the same for all phases of applied field as noted by a constant transit time between the electrodes independent of field polarity. However, the time to travel between the electrodes that span the field (R1-R2) is shorter during the application of a positive field (upper traces) and is longer during the application of a negative field (lower traces), relative to a zero field (middle traces).

The speed of propagation as a function of field is shown in FIG. 4B for two local field applications in different slices. In the top plots, for which the field was presented within the field (R1-R2) and after the activity wave leaves the field (R2-R3). As with the global field, the speed of propagation increased between R1 and R2 as the field amplitude became more positive and decreased as the field amplitude became more negative. Alternatively, there was no change in speed with field amplitude in the region outside the local field (R2-R3). Similar results were seen when the field was applied farther from the initiation site (FIG. 4B, bottom). Prior to entering the local field region, propagation speed was not dependent on the applied field (R1-R2). When the activity wave entered the local field area (R2-R3) the speed increased with increasing positive field amplitude and decreased with increasing negative field amplitude, as expected.

What we claim:

1. A method for suppressing the propagation of epileptiform activity in a mammalian brain, comprising:
applying an amount of a sub-threshold electric field to a mammalian brain experiencing ongoing epileptiform activity,
wherein said electric field is applied to a region of the brain which is not experiencing epileptiform activity and the amount is effective for suppressing the propagation of epileptiform activity in said mammalian brain.

2. A method of claim 1, wherein said electric field is applied to a region of the brain other than the focal region of the ongoing epileptiform activity.

3. A method of claim 1, wherein said electric field is applied to a region of the brain which is not experiencing epileptiform activity, and said electric field blocks the propagation of the epileptiform activity into that region of the brain.

4. A method of claim 1, wherein said electric field is from 5-125 mV per mm.

5. A method of claim 1, further comprising measuring changes in neuronal activity in said brain within a time period.

6. A method of claim 1, wherein the field is applied when an epileptiform activity is measured.

7. A method of claim 1, wherein said epileptiform activity is an epileptic seizure.

8. A method of claim 1, wherein the electric field is applied through field electrodes.

9. A method of claim 8, wherein the field electrodes are positioned in a region of the brain which is outside the epileptic focus.

11. A method of claim 1, wherein said applied field is a hyperpolarizing field.

12. A method of claim 1, wherein a plurality of electric field stimuli are applied.

13. A method for modulating the propagation speed of a traveling wave in a mammalian brain, comprising:
applying an amount of an electric field to a mammalian brain that is experiencing a traveling wave of electrical neuronal activity,
wherein said amount is effective for modulating the propagation speed and/or direction of a traveling wave in a mammalian brain.

14. A method of claim 13, wherein said applied field is a hyperpolarizing field.

15. A method of claim 13, where said applied field is sub-threshold.

16. A method of claim 13, wherein a plurality of electric field stimuli are applied.

17. A neural device for modulating the propagation speed of a traveling wave in a mammalian brain, comprising:
a field electrode set for producing an electric field;
a signal generator programmed to deliver a hyperpolarizing electric field to a mammalian brain, wherein said signal generator is operably connected to said field electrode set.
